(19) United States
(12) Patent Application Publication
(10) Pub. No.: US 2008/0214570 A1
(43) Pub. Date: Sep. 4, 2008

(54) Therapeutic Agents

(75) Inventors: James R. Empfield, Wilmington, DE (US); Michael Laplante, Montreal (CA); Megan Murphy King, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/997,438

(22) PCT Filed: Aug. 3, 2006
(86) PCT No.: PCT/SE2006/000926

§ 371 Date: Jan. 31, 2008

Related U.S. Application Data
(60) Provisional application No. 60/706,470, filed on Aug. 8, 2005.

(51) Int. Cl. A61K 31/495; C07D 295/26; A61P 25/00; A61P 25/18; A61P 3/04

(52) U.S. Cl. 514/255.03; 544/381

(57) ABSTRACT

The present invention relates to compounds of formula I and processes for preparing such compounds, their use in the treatment of obesity, psychiatric and neurological disorders, to methods for their therapeutic use and to pharmaceutical compositions containing them.

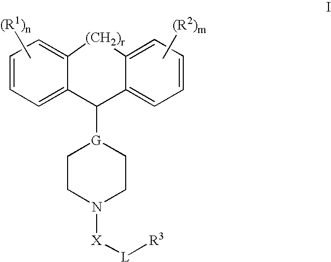

### FIELD OF INVENTION

The present invention relates to certain CB₁ antagonists or inverse agonists, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB₁ antagonists or inverse agonists (known as antagonists or inverse agonists) are useful in the treatment of obesity, psychiatric and neurological disorders (WO01/70700 and EP 656354).

However, there is a need for CB₁ antagonists or inverse agonists with other therapeutic effects, improved physicochemical properties and/or DMPK properties and/or pharmacodynamic properties.

### DESCRIPTION OF THE INVENTION

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable to substituents.

The term “cycloalkyl,” used alone or as suffix or prefix, refers to a saturated monovalent ring-containing hydrocarbon radical comprising at least 3 up to about 12 carbon atoms. Examples of cycloalkyls include, but are not limited to, C₃₋₇cycloalkyl groups, such as cyclopropyl, cyclobutyl, cyclopentyl, cyclohexyl, and cycloheptyl, and saturated cyclic and bicyclic terpenes. A cycloalkyl can be unsubstituted or substituted by one or two suitable substituents. Preferably, the cycloalkyl is a monocyclic ring or bicyclic ring.

The term “aryl” used alone or as suffix or prefix, refers to a monovalent hydrocarbon radical having one or more polyunsaturated carbon rings having aromatic character, (e.g., 4n+2 delocalized electrons) and comprising 5 up to about 14 carbon atoms.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heteroaryl” used alone or as a suffix or prefix, refers to a heterocyclyl having aromatic character.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

Halogen includes fluorine, chlorine, bromine and iodine.

“Halogenated,” used as a prefix of a group, means one or more hydrogens on the group is replaced with one or more halogens.

“RT” or “rt” means room temperature.

In one aspect, the invention provides a compound of formula I, and pharmaceutically acceptable salts thereof

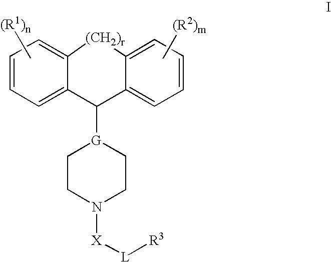

wherein

G is selected from CH and N;

r is selected from 0, 1 and 2;

R¹ and R² are independently selected from hydrogen, —OH, halogen, C₁₋₆alkyl, halogenated C₁₋₆alkyl, C₁₋₆alkoxy, halogenated C₁₋₆alkoxy, —NR⁴C(═O)—O—R⁴, —S(═O)₂—NR⁴R⁴ and —O—S(═O)₂—R⁴; wherein each R⁴ is independently selected from hydrogen, C₁₋₆alkyl, halogenated C₁₋₆alkyl, C₃₋₆cycloalkyl, and halogenated C₃₋₆cycloalkyl;

m and n are independently selected from 1, 2, and 3;

X is selected from a bond, —C(═O)—, —S(═O)₂—, —C(═O)—N—, and —C(═S)—N—;

L is selected from a bond, —(CH2)ₚ—, —(CH₂)ₚ—O—, —C(═O)—O—, —(CH2)ₚS—, —CH₂NHC(═O)—CH₂—,

p is selected from 0, 1 and 2; and

R³ is selected from C₁₋₆alkyl, C₃₋₆cycloalkyl, C₆₋₁₀aryl, and C₃₋₉-heterocyclyl, wherein said C₁₋₆alkyl, C₃₋₆cycloalkyl, C₆₋₁₀aryl, and C₃₋₉-heterocyclyl are optionally substituted with one or more group selected from C₁₋₆alkoxy, halogen, C₁₋₆alkyl, halogenated C₁₋₆alkyl, aryl, halogenated aryl, nitro, cyano, —C(═O)—R⁵, —CO₂R⁵, —NHC(═O)—R⁵, wherein R⁵ is selected from hydrogen, C₁₋₆alkyl, halogenated C₁₋₆alkyl, C₃₋₆cycloalkyl, halogenated C₃₋₆cycloalkyl, heterocyclyl, benzhydryl and aryl, wherein said heterocyclyl, benzhydryl and aryl are optionally substituted by one or more group selected from halogen, C₁₋₃alkyl, phenyl, and methoxy;

with a proviso that at least one of X and L is not a bond.

In one embodiment, the compounds of the present invention are represented by formula I, wherein

G is selected from CH and N;

r is selected from 0, 1 and 2;

R¹ and R² are independently selected from hydrogen, —OH, halogen, C₁₋₃alkyl, halogenated C₁₋₃alkyl, C₁₋₃alkoxy, halogenated C₁₋₃alkoxy, —NR⁴C(═O)—O—R⁴, —S(═O)₂—NR⁴R⁴ and —O—S(═O)₂—R⁴; wherein each R⁴ is independently selected from hydrogen and C₁₋₃alkyl;

m and n are independently selected from 1 and 2;

X is selected from a bond, —C(═O)—, —S(═O)₂—, —C(═O)—N—, and —C(═S)—N—;

L is selected from a bond, —(CH2)ₚ—, —C(═O)—O—, —(CH₂)ₚ—O—, —(CH2)ₚS—, —CH₂NHC(═O)—CH₂—,

p is selected from 1 and 2;

R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from C₁₋₃alkoxy, halogen, C₁₋₃alkyl, halogenated C₁₋₃alkyl, C₆₋₁₀aryl, halogenated C₆₋₁₀-aryl, nitro, cyano, —C(═O)—R⁵, —CO₂R⁵, —NHC(═O)—R⁵, wherein R⁵ is selected from hydrogen, C₁₋₃alkyl, halogenated C₁₋₃alkyl, C₃₋₆cycloalkyl, halogenated C₃₋₆cycloalkyl, heterocyclyl, benzhydryl and phenyl, wherein said heterocyclyl, benzhydryl and aryl are optionally substituted by one or more group selected from halogen, C₁₋₃alkyl, phenyl, and methoxy;

with a proviso that R³ is not unsubstituted phenyl; with a further proviso that at least one of X and L is not a bond.

In another embodiment, the compound of the invention may be represented by formula I, wherein

G is selected from CH and N;

r is selected from 0, 1 and 2;

R¹ and R² are independently selected from hydrogen, —OH, halogen, C₁₋₃alkyl, halogenated C₁₋₃alkyl, C₁₋₃alkoxy, and halogenated C₁₋₃alkoxy;

m and n are independently selected from 1 and 2;

X is selected from a bond, —C(═O)—, and —S(═O)₂—;

L is selected from a bond and —(CH2)ₚ—,

p is selected from 1 and 2;

R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from C₁₋₃alkoxy, halogen, cyano, C₁₋₃alkyl, halogenated C₁₋₃alkyl, and benzhydryl;

with a proviso that R³ is not unsubstituted phenyl; with a further proviso that at least one of X and L is not a bond.

In a further embodiment, X is —C(═O)—.

In an even further embodiment, L is selected from a bond and —CH₂—.

In a yet even further embodiment, R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from fluoro, chloro, bromo, nitro, methyl, trifluoromethyl, cyano, methoxy, and benzhydryl.

In a further embodiment, compounds of the invention may be represented by formula I, wherein

G is selected from CH and N;

r is selected from 0, 1 and 2;

R¹ and R² are independently selected from hydrogen, fluoro, chloro, bromo, methyl, trifluoromethyl, and methoxy;

m and n are independently selected from 1 and 2;

X is —C(═O)—;

L is selected from a bond and —CH₂—,

R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from fluoro, chloro, bromo, nitro, methyl, trifluoromethyl, cyano, methoxy, and benzhydryl;

with a proviso that R³ is not unsubstituted phenyl.

In one aspect, the invention provides a compound of formula IA, and pharmaceutically acceptable salts thereof

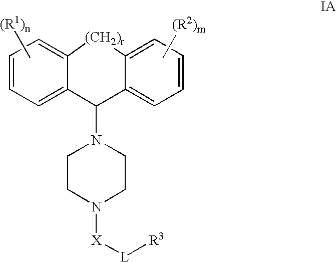

wherein

R¹ and R² are independently selected from hydrogen, —OH, halogen, C₁₋₆alkyl, halogenated C₁₋₆alkyl, C₁₋₆alkoxy, halogenated C₁₋₆alkoxy, —NR⁴C(═O)—O—R⁴, —S(═O)₂—NR⁴R⁴ and —O—S(═O)₂—R⁴; wherein each R⁴ is independently selected from hydrogen, C₁₋₆allyl, halogenated C₁₋₆alkyl, C₃₋₆cycloalkyl, and halogenated C₃₋₆cycloalkyl;

r is selected from 0, 1 and 2;

m and n are independently selected from 1, 2, 3, 4 and 5;

X is selected from a bond, —C(═O)—, —S(═O)₂—, —C(═O)—N—, and —C(═S)—N—;

L is selected from a bond, —(CH2)ₚ—, —(CH₂)ₚ—O—, —C(═O)—O—, —(CH2)ₚS—, —CH₂NHC(═O)—CH₂—,

p is selected from 0, 1 and 2;

R³ is selected from C₁₋₆alkyl, C₃₋₆cycloalkyl, C₆₋₁₀aryl, and C₃₋₉heterocyclyl, wherein said C₁₋₆alkyl, C₃₋₆cycloalkyl, C₆₋₁₀aryl, and C₃₋₉-heterocyclyl are optionally substituted with one or more group selected from C₁₋₆alkoxy, halogen, C₁₋₆alkyl, halogenated C₁₋₆alkyl, aryl, halogenated aryl, nitro, cyano, —C(═O)—R⁵, —CO₂R⁵, —NHC(═O)—R⁵, wherein R⁵ is selected from hydrogen, C₁₋₆alkyl, halogenated C₁₋₆alkyl, C₃₋₆cycloalkyl, halogenated C₃₋₆cycloalkyl, heterocyclyl, benzhydryl and aryl, wherein said heterocyclyl, benzhydryl and aryl are optionally substituted by one or more group selected from halogen, C₁₋₃alkyl, phenyl, and methoxy;

with a proviso that at least one of X and L is not a bond;

In one embodiment, the compounds of the present invention are represented by formula IA, wherein

R¹ and R² are independently selected from hydrogen, —OH, halogen, C₁₋₃alkyl, halogenated C₁₋₃alkyl, C₁₋₃alkoxy, halogenated C₁₋₃alkoxy, —NR⁴C(═O)—O—R⁴, —S(═O)₂—NR⁴R⁴ and —O—S(═O)₂—R⁴; wherein each R⁴ is independently selected from hydrogen and C₁₋₃alkyl;

r is selected from 0, 1 and 2;

m and n are independently selected from 1, 2 and 3;

X is selected from a bond, —C(═O)—, —S(═O)₂—, —C(═O)—N—, and —C(═S)—N—;

L is selected from a bond, —(CH2)ₚ—, —C(═O)—O—, —(CH₂)ₚ—O—, —(CH2)ₚS—, —CH₂NHC(═O)—CH₂—,

p is selected from 1 and 2;

R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from C₁₋₃alkoxy, halogen, C₁₋₃alkyl, halogenated C₁₋₃alkyl, C₆₋₁₀aryl, halogenated C₆₋₁₀aryl, nitro, cyano, —C(═O)—R⁵, —CO₂R⁵, —NHC(═O)—R⁵, wherein R⁵ is selected from hydrogen, C₁₋₃alkyl, halogenated C₁₋₃alkyl, C₃₋₆cycloalkyl, halogenated C₃₋₆cycloalkyl, heterocyclyl, benzhydryl and phenyl, wherein said heterocyclyl, benzhydryl and aryl are optionally substituted by one or more group selected from halogen, C₁₋₃alkyl, phenyl, and methoxy;

with a proviso that R³ is not unsubstituted phenyl; with a further proviso that at least one of X and L is not a bond.

In another embodiment, the compound of the invention may be represented by formula IA, wherein

R¹ and R² are independently selected from hydrogen, —OH, halogen, C₁₋₃alkyl, halogenated C₁₋₃alkyl, C₁₋₃alkoxy, and halogenated C₁₋₃alkoxy;

r is selected from 0, 1 and 2;

m and n are independently selected from 1 and 2;

X is selected from a bond, —C(═O)—, and —S(═O)₂—;

L is selected from a bond and —(CH2)ₚ—,

p is selected from 1 and 2;

R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from C₁₋₃alkoxy, halogen, nitro, cyano, C₁₋₃alkyl, halogenated C₁₋₃alkyl, and benzhydryl;

with a proviso that R³ is not unsubstituted phenyl; with a further proviso that at least one of X and L is not a bond.

In a further embodiment, X is —C(═O)—.

In an even further embodiment, L is selected from a bond and —CH₂—.

In a yet even further embodiment, R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from fluoro, chloro, bromo, methyl, trifluoromethyl, cyano, methoxy, and benzhydryl.

In a further embodiment, compounds of the invention may be represented by formula IA, wherein

R¹ and R² are independently selected from hydrogen, fluoro, chloro, bromo, methyl, trifluoromethyl, and methoxy;

r is selected from 0, 1 and 2;

m and n are independently selected from 1 and 2;

X is —C(═O)—;

L is selected from a bond and —CH₂—,

R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from fluoro, chloro, bromo, nitro, methyl, trifluoromethyl, cyano, methoxy, and benzhydryl; and

with a proviso that R³ is not unsubstituted phenyl.

In a further embodiment, the invention provides a compound of formula IB or pharmaceutically acceptable salts thereof:

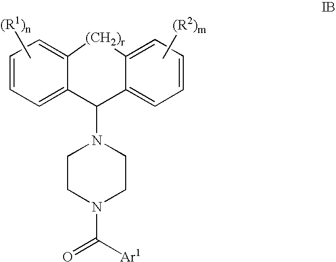

wherein

R¹ and R² are independently selected from hydrogen, fluoro, chloro, bromo, methyl, trifluoromethyl, and methoxy;

r is selected from 0, 1 and 2;

m and n are independently selected from 1 and 2; and

Ar¹ is selected from phenyl, and C₃₋₅heteroaryl, wherein said phenyl, and C₃₋₅heteroaryl are substituted with one or more group selected from fluoro, chloro, bromo, nitro, methyl, trifluoromethyl, cyano, methoxy, and benzhydryl.

In an even further embodiment, the invention provides a compound of formula IC or pharmaceutically acceptable salts thereof:

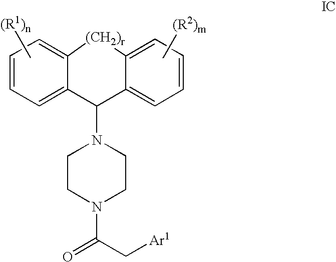

wherein

R¹ and R² are independently selected from hydrogen, fluoro, chloro, bromo, methyl, trifluoromethyl, and methoxy;

r is selected from 0, 1 and 2;

m and n are independently selected from 1 and 2; and

Ar¹ is selected from phenyl, and C₃₋₅heteroaryl, wherein said phenyl, and C₃₋₅heteroaryl are substituted with one or more group selected from fluoro, chloro, bromo, nitro, methyl, trifluoromethyl, cyano, methoxy, and benzhydryl.

“Pharmaceutically acceptable salt”, where such salts are possible, includes both pharmaceutically acceptable acid and base addition salts. A suitable pharmaceutically acceptable salt of a compound of Formula I, IA, IB or IC is, for example, an acid-addition salt of a compound of Formula I, IA, IB or IC which is sufficiently basic, for example an acid-addition salt with an inorganic or organic acid such as hydrochloric, hydrobromic, sulphuric, trifluoroacetic, citric or maleic acid; or, for example a salt of a compound of Formula I, IA, IB or IC which is sufficiently acidic, for example an alkali or alkaline earth metal salt such as a sodium, calcium or magnesium salt, or an ammonium salt, or a salt with an organic base such as methylamine, dimethylamine, trimethylamine, piperidine, morpholine or tris-(2-hydroxyethyl)amine.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention. All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies.

The present invention also encompasses prodrugs of a compound of formula I, IA, IB or IC that is compounds which are converted into a compound of formula I, IA, IB or IC in vivo.

### Methods of Preparation

The compounds of the invention may be prepared as outlined below according to any of the following methods. However, the invention is not limited to these methods, the compounds may also be prepared as described for structurally related compounds in the prior art.

Compounds of formula I, wherein R¹, R², R³, r, n, m, G, X and L are as defined above, may be prepared by reacting a compound of formula IIA

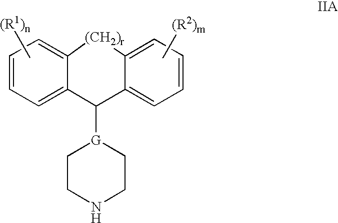

with Y—X-L-R³, wherein Y represents a leaving group, for example, halo or —OH, at a temperature in the range of −25 to 150° C., in the presence of an inert solvent, for example dichloromethane, and optionally in the presence of a base for example triethylamine or pyridine, and optionally in the presence of DMAP.

Compounds of formula IA, wherein R¹, R², R³, r, n, m, X and L are as defined above, may be prepared by reacting a compound of formula II

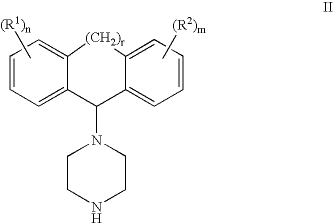

with Y—X-L-R³, wherein Y represents a leaving group, for example, halo or —OH, at a temperature in the range of −25 to 150° C., in the presence of an inert solvent, for example dichloromethane, and optionally in the presence of a base for example triethylamine or pyridine, and optionally in the presence of DMAP.

Compounds of formula IB, wherein R¹ and R² are independently selected from hydrogen, fluoro, chloro, bromo, methyl, trifluoromethyl, and methoxy;

r is selected from 0, 1 and 2;

m and n are independently selected from 1 and 2; and

Ar¹ is selected from phenyl, and C₃₋₅heteroaryl, wherein said phenyl, and C₃₋₅heteroaryl are substituted with one or more group selected from fluoro, chloro, bromo, nitro, methyl, trifluoromethyl, cyano, methoxy, and benzhydryl,

may be prepared by reacting a compound of formula II

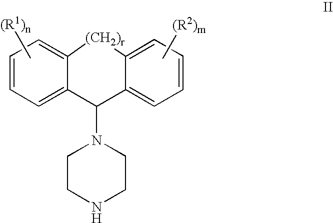

with Y—C(═O)—Ar¹, wherein Y represents a leaving group, for example, halo or —OH, at a temperature in the range of −25 to 150° C., in the presence of an inert solvent, for example dichloromethane, and optionally in the presence of a base for example triethylamine or pyridine, and optionally in the presence of DMAP.

Compounds of formula IC, wherein R¹ and R² are independently selected from hydrogen, fluoro, chloro, bromo, methyl, trifluoromethyl, and methoxy;

r is selected from 0, 1 and 2;

m and n are independently selected from 1 and 2; and

Ar¹ is selected from phenyl, and C₃₋₅heteroaryl, wherein said phenyl, and C₃₋₅heteroaryl are substituted with one or more group selected from fluoro, chloro, bromo, nitro, methyl, trifluoromethyl, cyano, methoxy, and benzhydryl,

may be prepared by reacting a compound of formula II

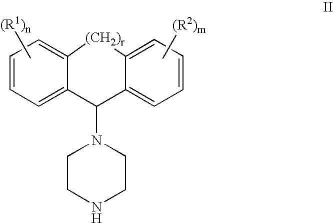

with Y—C(═O)—Ar¹, wherein Y represents a leaving group, for example, halo or —OH, at a temperature in the range of −25 to 150° C., in the presence of an inert solvent, for example dichloromethane, and optionally in the presence of a base for example triethylamine or pyridine, and optionally in the presence of DMAP.

Compounds of formula II or IIA may be prepared by the general synthetic route shown at the end of the examples and adaptations thereof or by analogous methods known to those skilled in the art. It will be appreciated by those skilled in the art that during the reaction sequence certain functional groups will require protection followed by deprotection at an appropriate stage, see “Protective Groups in Organic Synthesis”, 3rd Edition (1999) by Greene and Wuts.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

### Pharmacological Properties

The compounds of formula I, IA, IB or IC are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia, cognitive deficiency associated with schizophrenia, schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

The compounds are also potentially useful for the prevention or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

The compounds are also potentially useful for the prevention or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

The compounds are also potentially useful for the treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

The compounds are also potentially useful as agents in treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders. The compounds are also potentially useful as agents in treatment of (esophageal) achalasia.

In another aspect the present invention provides a compound of formula I, IA, IB or IC as previously defined for use as a medicament.

In a further aspect the present invention provides the use of a compound of formula I, IA, IB or IC in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia, cognitive deficiency associated with schizophrenia, cognitive deficiency associated with schizophrenia, schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a further aspect the present invention provides the use of a compound of formula I, IA, IB or IC in the preparation of a medicament for the treatment or prophylaxis of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a further aspect the present invention provides the use of a compound of formula I, IA, IB or IC in the preparation of a medicament for the treatment or prophylaxis of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a further aspect the present invention provides the use of a compound of formula I, IA, IB or IC in the preparation of a medicament for the treatment or prophylaxis of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a further aspect the present invention provides the use of a compound of formula I, IA, IB or IC in the preparation of a medicament for the treatment or prophylaxis of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

In a still further aspect the present invention provides a method comprising administering a pharmacologically effective amount of a compound of formula I, IA, IB or IC to a patient in need thereof for the prophylaxis or treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia, cognitive deficiency associated with schizophrenia, schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a still further aspect the present invention provides a method comprising administering a pharmacologically effective amount of a compound of formula I, IA, IB or IC to a patient in need thereof for the prophylaxis or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a still further aspect the present invention provides a method comprising administering a pharmacologically effective amount of a compound of formula I, IA, IB or IC to a patient in need thereof for the prophylaxis or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a still further aspect the present invention provides a method comprising administering a pharmacologically effective amount of a compound of formula I, IA, IB or IC to a patient in need thereof for the prophylaxis or treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic chock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a still further aspect the present invention provides a method comprising administering a pharmacologically effective amount of a compound of formula I, IA, IB or IC to a patient in need thereof for the prophylaxis or treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

The compounds of the present invention are particularly suitable for the treatment of obesity or being overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items).

The compounds of formula I, IA, IB or IC are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders (e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight that normally accompanies the cessation of smoking.

In another aspect the present invention provides a compound of formula I, IA, IB or IC as previously defined for use as a medicament.

In a further aspect the present invention provides the use of a compound of formula I, IA, IB or IC in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I, IA, IB or IC to a patient in need thereof.

The compounds of the present invention are particularly suitable for the treatment of obesity, e.g. by reduction of appetite and body weight, maintenance of weight reduction and prevention of rebound.

The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation.

The compounds of the present invention are suitable for use in treating the above indications in juvenile or adolescent patient populations.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-I motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, IA, IB or IC, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula I, IA, IB or IC, or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;

a cholesterol absorption antagonist;

a MTP (microsomal transfer protein) inhibitor;

a nicotinic acid derivative, including slow release and combination products;

a phytosterol compound;

probucol;

an anti-coagulant;

an omega-3 fatty acid;

another anti-obesity compound for example sibutramine, phentermine, orlistat, bupropion, ephedrine, thyroxine;

an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;

a melanin concentrating hormone (MCH) modulator;

an NPY receptor modulator;

an orexin receptor modulator;

a phosphoinositide-dependent protein kinase (PDK) modulator; or modulators of nuclear receptors for example LXR, FXR, RXR, GR, ERRα, β, PPARα, β, γ and RORalpha;

a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);

an antipsychotic agent for example olanzapine and clozapine;

a serotonin receptor modulator;

a leptin/leptin receptor modulator;

a ghrelin/ghrelin receptor modulator;

a DPP-IV inhibitor;

or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula I, IA, IB or IC, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of formula I, IA, IB or IC, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, IA, IB or IC, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, IA, IB or IC, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, IA, IB or IC, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, IA, ID or IC, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.
According to a further aspect of the present invention there is provided a kit comprising:
a) a compound of formula I, IA, IB or IC, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, IA, IB or IC, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, IA, IB or IC, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, IA, IB or IC, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

### Pharmacological Activity

Compounds of the present invention are active against the receptor product of the CB1 gene. The affinity of the compounds of the invention for central cannabinoid receptors is demonstrable in methods described in Devane et al, Molecular Pharmacology, 1988, 34,605 or those described in WO01/70700 or EP 656354. Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS.

The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x)UD)) and the IC50 value determined as the concentration required to give half maximal inhibition of GTPγS binding under the conditions used.

The compounds of the present invention are active at the CB1 receptor (IC50<1 micromolar). Most preferred compounds have IC50<200 nanomolar.

The compounds of the invention are believed to be selective CB1 antagonists or inverse agonists. The potency, selectivity profile and side effect propensity may limit the clinical usefulness of hitherto known compounds with alleged CB1 antagonistic/inverse agonistic properties. In this regard, preclinical evaluation of compounds of the present invention in models of gastrointestinal and/or cardiovascular function indicates that they offer significant advantages compared to representative reference CB1 antagonist/inverse agonist agents.

The compounds of the present invention may provide additional benefits in terms of potency, selectivity profile, bioavailability, half-life in plasma, blood brain permeability, plasma protein binding (for example increasing the free fraction of drug) or solubility compared to representative reference CB1 antagonists/inverse agonist agents.

The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks. Compounds to be tested were then administered systemically (iv, ip, sc or po) once daily for a minimum of 5 days, and the body weights of the mice monitored on a daily basis. Simultaneous assessment of adiposity was carried by means of DEXA imaging at baseline and termination of the study. Blood sampling was also carried out to assay changes in obesity-related plasma markers.

### EXAMPLES

### Abbreviations

DMF dimethylformamide

### DMSO Dimethyl Sulfoxide

### DEA Diethylamine

EtOAc ethyl acetate
THF tetrahydrofuran

### DMAP 4-Dimethylaminopyridine

TLC thin layer chromatography
t triplet
singlet
d doublet
q quartet
qvint quintet
m multiplet
br broad
bs broad singlet
dm doublet of multiplet
bt broad triplet
dd doublet of doublet

### Example 1

### 1-(10,11-Dihydro-5H-dibenzo[a,d]cyclohepten-5-yl)-4-(2-nitro-phenylmethanesulfonyl)-piperazine

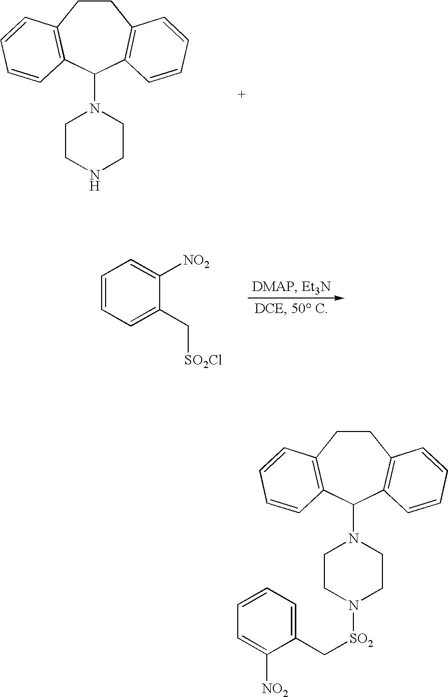

To a solution of 1-(dibenzosuberyl)-piperazine (0.118 g, 0.42 mmol) in 1,2-dichloroethane (10 mL) was added a solution of 2-nitro-α-toluenesulfonyl chloride (2.8 mL of a 0.15 M 1,2-dichloroethane solution, 0.42 mmol), triethylamine (69 μL, 0.50 mmol) and DMAP (5 mg, 0.042 mmol). The mixture was stirred at 50° C. overnight, then evaporated to dryness (100 mg). The crude compound was redissolved in DMF (2 mL) and purified by prep LC/MS to yield 67 mg (33%) of the title compound as the TFA salt. +ES MS (M+1) 478.2.

1. A compound of formula IA or pharmaceutically acceptable salts thereof 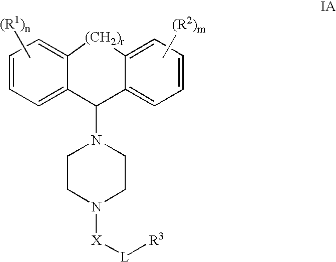
wherein
R¹ and R² are independently selected from hydrogen, —OH, halogen, C₁₋₆alkyl, halogenated C₁₋₆alkyl, C₁₋₆alkoxy, halogenated C₁₋₆alkoxy, —NR⁴C(═O)—O—R⁴, —S(═O)₂—NR⁴R⁴ and —O—S(═O)₂—R⁴; wherein each R⁴ is independently selected from hydrogen, C₁₋₆alkyl, halogenated C₁₋₆alkyl, C₃₋₆cycloalkyl, and halogenated C₃₋₆cycloalkyl;
r is selected from 0, 1 and 2;
m and n are independently selected from 1, 2, 3 and 4;
X is selected from a bond, —C(═O)—, —S(═O)₂—, —C(═O)—N—, and —C(═S)—N—;
L is selected from a bond, —(CH2)ₚ—, —(CH₂)ₚ—O—, —C(═O)—O—, —(CH2)ₚS—, —CH₂NHC(═O)—CH₂—,
p is selected from 0, 1 and 2;
R³ is selected from C₁₋₆alkyl, C₃₋₆cycloalkyl, C₆₋₁₀aryl, and C₃₋₉heterocyclyl, wherein said C₁₋₆alkyl, C₃₋₆cycloalkyl, C₆₋₁₀aryl, and C₃₋₉heterocyclyl are optionally substituted with one or more group selected from C₁₋₆alkoxy, halogen, C₁₋₆alkyl, halogenated C₁₋₆alkyl, aryl, halogenated aryl, nitro, cyano, —C(═O)—R⁵, —CO₂R⁵, —NHC(═O)—R⁵, wherein R⁵ is selected from hydrogen, C₁₋₆alkyl, halogenated C₁₋₆alkyl, C₃₋₆cycloalkyl, halogenated C₃₋₆cycloalkyl, heterocyclyl, benzhydryl and aryl, wherein said heterocyclyl, benzhydryl and aryl are optionally substituted by one or more group selected from halogen, C₁₋₃alkyl, phenyl, and methoxy;
with a proviso that at least one of X and L is not a bond.

2. A compound as claimed in claim 1, wherein
R¹ and R² are independently selected from hydrogen, —OH, halogen, C₁₋₃alkyl, halogenated C₁₋₃alkyl, C₁₋₃alkoxy, halogenated C₁₋₃alkoxy, —NR⁴C(═O)—O—R⁴, —S(═O)₂—NR⁴R⁴ and —O—S(═O)₂—R⁴; wherein each R⁴ is independently selected from hydrogen and C₁₋₃alkyl;
m and n are independently selected from 1, 2 and 3;
X is selected from a bond, —C(═O)—, —S(═O)₂—, —C(═O)—N—, and —C(═S)—N—;
L is selected from a bond, —(CH2)ₚ—, —C(═O)—O—, —(CH₂)ₚ—O—, —(CH2)ₚS—, —CH₂NHC(═O)—CH₂—,
p is selected from 1 and 2;
R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from C₁₋₃alkoxy, halogen, C₁₋₃alkyl, halogenated C₁₋₃alkyl, C₆₋₁₀aryl, halogenated C₆₋₁₀aryl, nitro, cyano, —C(═O)—R⁵, —CO₂R⁵, —NHC(═O)—R⁵, wherein R⁵ is selected from hydrogen, C₁₋₃alkyl, halogenated C₁₋₃alkyl, C₃₋₆cycloalkyl, halogenated C₃₋₆cycloalkyl, heterocyclyl, benzhydryl and phenyl, wherein said heterocyclyl, benzhydryl and aryl are optionally substituted by one or more group selected from halogen, C₁₋₃alkyl, phenyl, and methoxy;
with a further proviso that R³ is not unsubstituted phenyl.

3. A compound as claimed in claim 1, wherein
R¹ and R² are independently selected from hydrogen, —OH, halogen, C₁₋₃alkyl, halogenated C₁₋₃alkyl, C₁₋₃alkoxy, and halogenated C₁₋₃alkoxy;
m and n are independently selected from 1 and 2;
X is selected from a bond, —C(═O)—, and —S(═O)₂—;
L is selected from a bond and —(CH2)ₚ—,
p is selected from 1 and 2;
R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from C₁₋₃alkoxy, halogen, nitro, cyano, C₁₋₃alkyl, halogenated C₁₋₃alkyl, and benzhydryl;
with a proviso that R³ is not unsubstituted phenyl.

4. A compound as claimed in claim 1, wherein
R¹ and R² are independently selected from hydrogen, fluoro, chloro, bromo, methyl, trifluoromethyl, and methoxy.

5. A compound as claimed in claim 1, wherein X is —C(═O)—.

6. A compound as claimed in claim 1, wherein L is selected from a bond and —CH₂—.

7. A compound as claimed in claim 1, wherein R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from fluoro, chloro, bromo, nitro, methyl, trifluoromethyl, cyano, methoxy, and benzhydryl.

8. A compound as claimed in claim 1, wherein
R¹ and R² are independently selected from hydrogen, fluoro, chloro, bromo, methyl, trifluoromethyl, and methoxy;
m and n are independently selected from 1 and 2;
X is —C(═O)—;
L is selected from a bond and —CH₂—,
R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from fluoro, chloro, bromo, nitro, methyl, trifluoromethyl, cyano, methoxy, and benzhydryl.

9. A compound of formula IB or pharmaceutically acceptable salts thereof: 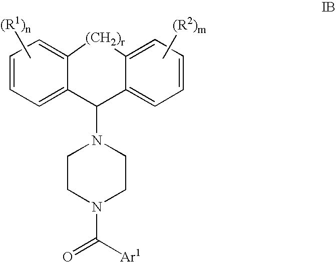
wherein
R¹ and R² are independently selected from hydrogen, fluoro, chloro, bromo, methyl, trifluoromethyl, and methoxy;
r is selected from 0, 1 and 2;
m and n are independently selected from 1 and 2;
Ar¹ is selected from phenyl, and C₃₋₅heteroaryl, wherein said phenyl, and C₃₋₅heteroaryl are substituted with one or more group selected from fluoro, chloro, bromo, nitro, methyl, trifluoromethyl, cyano, methoxy, and benzhydryl.

10. A compound of formula IC or pharmaceutically acceptable salts thereof: 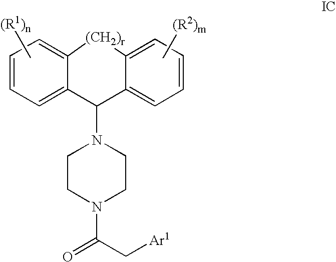
wherein
R¹ and R² are independently selected from hydrogen, fluoro, chloro, bromo, methyl, trifluoromethyl, and methoxy;
r is selected from 0, 1 and 2;
m and n are independently selected from 1 and 2;
Ar¹ is selected from phenyl, and C₃₋₅heteroaryl, wherein said phenyl, and C₃₋₅heteroaryl are substituted with one or more group selected from fluoro, chloro, bromo, nitro, methyl, trifluoromethyl, cyano, methoxy, and benzhydryl.

11. A compound selected from 1-(10,11-Dihydro-5H-dibenzo[a,d]cyclohepten-5-yl)-4-(2-nitro-phenylmethanesulfonyl)-piperazine and pharmaceutically acceptable salts thereof.

12. A compound of formula I or pharmaceutically acceptable salts thereof 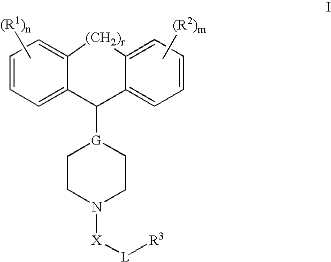
wherein
G is selected from CH and N;
R¹ and R² are independently selected from hydrogen, —OH, halogen, C₁₋₆alkyl, halogenated C₁₋₆alkyl, C₁₋₆alkoxy, halogenated C₁₋₆alkoxy, —NR⁴C(═O)—O—R⁴, —S(═O)₂—NR⁴R⁴ and —O—S(═O)₂—R⁴; wherein each R⁴ is independently selected from hydrogen, C₁₋₆alkyl, halogenated C₁₋₆alkyl, C₃₋₆cycloalkyl, and halogenated C₃₋₆cycloalkyl;
r is selected from 0, 1 and 2;
m and n are independently selected from 1, 2, 3 and 4;
X is selected from a bond, —C(═O)—, —S(═O)₂—, —C(═O)—N—, and —C(═S)—N—;
L is selected from a bond, —(CH2)ₚ—, —(CH₂)ₚ—O—, —C(═O)—O—, —(CH2)ₚS—, —CH₂NHC(═O)—CH₂—,
p is selected from 0, 1 and 2;
R³ is selected from C₁₋₆alkyl, C₃₋₆cycloalkyl, C₆₋₁₀aryl, and C₃₋₉heterocyclyl, wherein said C₁₋₆alkyl, C₃₋₆cycloalkyl, C₆₋₁₀aryl, and C₃₋₉heterocyclyl are optionally substituted with one or more group selected from C₁₋₆alkoxy, halogen, C₁₋₆alkyl, halogenated C₁₋₆alkyl, aryl, halogenated aryl, nitro, cyano, —C(═O)—R⁵, —CO₂R⁵, —NHC(═O)—R⁵, wherein R⁵ is selected from hydrogen, C₁₋₆alkyl, halogenated C₁₋₆alkyl, C₃₋₆cycloalkyl, halogenated C₃₋₆cycloalkyl, heterocyclyl, benzhydryl and aryl, wherein said heterocyclyl, benzhydryl and aryl are optionally substituted by one or more group selected from halogen, C₁₋₃alkyl, phenyl, and methoxy;
with a proviso that at least one of X and L is not a bond.

13. A compound as claimed in claim 12, wherein
R¹ and R² are independently selected from hydrogen, —OH, halogen, C₁₋₃alkyl, halogenated C₁₋₃alkyl, C₁₋₃alkoxy, halogenated C₁₋₃alkoxy, —NR⁴C(═O)—O—R⁴, —S(═O)₂—NR⁴R⁴ and —O—S(═O)₂—R⁴; wherein each R⁴ is independently selected from hydrogen and C₁₋₃alkyl;
m and n are independently selected from 1 and 2;
X is selected from a bond, —C(═O)—, —S(═O)₂—, —C(═O)—N—, and —C(═S)—N—;
L is selected from a bond, —(CH2)ₚ—, —C(═O)—O—, —(CH₂)ₚ—O—, —(CH2)ₚS—, —CH₂NHC(═O)—CH₂—,
p is selected from 1 and 2;
R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from C₁₋₃alkoxy, halogen, C₁₋₃alkyl, halogenated C₁₋₃alkyl, C₆₋₁₀aryl, halogenated C₆₋₁₀aryl, nitro, cyano, —C(═O)—R⁵, —CO₂R⁵, —NHC(═O)—R⁵, wherein R⁵ is selected from hydrogen, C₁₋₃alkyl, halogenated C₁₋₃alkyl, C₃₋₆cycloalkyl, halogenated C₃₋₆cycloalkyl, heterocyclyl, benzhydryl and phenyl, wherein said heterocyclyl, benzhydryl and aryl are optionally substituted by one or more group selected from halogen, C₁₋₃alkyl, phenyl, and methoxy;
with a further proviso that R³ is not unsubstituted phenyl.

14. A compound as claimed in claim 12, wherein
R¹ and R² are independently selected from hydrogen, —OH, halogen, C₁₋₃alkyl, halogenated C₁₋₃alkyl, C₁₋₃alkoxy, and halogenated C₁₋₃alkoxy;
m and n are independently selected from 1 and 2;
X is selected from a bond, —C(═O)—, and —S(═O)₂—;
L is selected from a bond and —(CH2)ₚ—,
p is selected from 1 and 2;
R³ is selected from C₁₋₆alkyl, C₃₋₆cycloalkyl, phenyl, and C₃₋₅heteroaryl, wherein said C₁₋₆alkyl, C₃₋₆cycloalkyl, phenyl, and C₃₋₅heteroaryl are optionally substituted with one or more group selected from C₁₋₃alkoxy, halogen, nitro, cyano, C₁₋₃alkyl, halogenated C₁₋₃alkyl, and benzhydryl;
with a further proviso that R³ is not unsubstituted phenyl.

15. (canceled)

16. A pharmaceutical formulation comprising a compound according to claim 1 and a pharmaceutically acceptable adjuvant, diluent or carrier.

17. (canceled)

18. A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, neurological disorders, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and/or relapse indications, comprising administering a pharmacologically effective amount of a compound according to claim 1 to a patient in need thereof.

19. A method of treating schizophrenia comprising administering a pharmacologically effective amount of a compound as claimed in claim 1 to a patient in need thereof.

20. A method of treating cognitive deficiency associated with schizophrenia comprising administering a pharmacologically effective amount of a compound as claimed in claim 1 to a patient in need thereof.

21-23. (canceled)

24. A process for the preparation of a compound of formula IA, 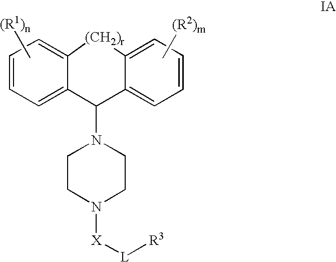
comprising reacting a compound of formula II 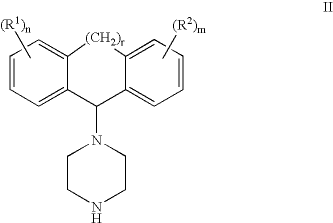
with Y—X-L-R³,
wherein
Y represents a leaving group,
R¹ and R² are independently selected from hydrogen, —OH, halogen, C₁₋₆alkyl, halogenated C₁₋₆alkyl, C₁₋₆alkoxy, halogenated C₁₋₆alkoxy, —NR⁴C(═O)—O—R⁴, —S(═O)₂—NR⁴R⁴ and —O—S(═O)₂—R⁴; wherein each R⁴ is independently selected from hydrogen, C₁₋₆alkyl, halogenated C₁₋₆alkyl, C₃₋₆cycloalkyl, and halogenated C₃₋₆cycloalkyl;
m and n are independently selected from 1, 2, 3 and 4;
X is selected from a bond, —C(═O)—, —S(═O)₂—, —C(═O)—N—, and —C(═S)—N—;
L is selected from a bond, —(CH2)ₚ—, —(CH₂)ₚ—O—, —C(═O)—O—, —(CH2)ₚS—, —CH₂NHC(═O)—CH₂—,
p is selected from 0, 1 and 2;
R³ is selected from C₁₋₆alkyl, C₃₋₆cycloalkyl, C₆₋₁₀aryl, and C₃₋₉heterocyclyl, wherein said C₁₋₆alkyl, C₃₋₆cycloalkyl, C₆₋₁₀aryl, and C₃₋₉heterocyclyl are optionally substituted with one or more group selected from C₁₋₆alkoxy, halogen, C₁₋₆alkyl, halogenated C₁₋₆alkyl, aryl, halogenated aryl, nitro, cyano, —C(═O)—R⁵, —CO₂R⁵, —NHC(═O)—R⁵, wherein R⁵ is selected from hydrogen, C₁₋₆alkyl, halogenated C₁₋₆alkyl, C₃₋₆cycloalkyl, halogenated C₃₋₆cycloalkyl, heterocyclyl, benzhydryl and aryl, wherein said heterocyclyl, benzhydryl and aryl are optionally substituted by one or more group selected from halogen, C₁₋₃alkyl, phenyl, and methoxy.
